(19) United States
(12) Patent Application Publication
(10) Pub. No.: US 2025/0032379 A1
(43) Pub. Date: Jan. 30, 2025

(54) STABLE COMPOSITION COMPRISING OIL AND WATER-SOLUBLE ALCOHOL

(71) Applicant: L'OREAL, Paris (FR)

(72) Inventors: Nathalie VARELA MONTEIRO, Saint-Ouen (FR); Adrien KAESER, Kawasaki-shi (JP); Yi-Yun TSAO, Kawasaki-shi (JP)

(21) Appl. No.: 18/718,966

(22) PCT Filed: Oct. 28, 2022
(86) PCT No.: PCT/JP2022/041275

§ 371 Date: Jun. 12, 2024

(30) Foreign Application Priority Data
Dec. 17, 2021 (JP) 2021-205209
Jan. 19, 2022 (FR) FR2200447

(51) Int. Cl. A61K 8/37; A61K 8/34; A61Q 5/00; A61Q 19/00

(52) U.S. Cl. CPC A61K 8/37; A61K 8/342; A61K 8/345; A61Q 5/00; A61Q 19/00

(57) ABSTRACT

A composition contains (a) at least one fatty alcohol, (b) at least one fatty acid ester oil, (c) at least one C₂-C₄ diol, (d) water in an amount of 35% by weight or less relative to the total weight of the composition. A total amount of oil(s) is less than 15% by weight, relative to the total weight of the composition.

### TECHNICAL FIELD

The present invention relates to a composition comprising an oil and a water-soluble alcohol, in particular a stable composition for keratinous substances, comprising the same.

### BACKGROUND ART

In the cosmetics field, cosmetic compositions making it possible to obtain care effects are increasingly sought after by users. In order to obtain care effects, active agents are generally included in the compositions. In cosmetic compositions including cosmetic active agents for keratinous substances, an oil can be used as a medium in the formulation in order to achieve a good penetration property of the active agents into keratinous substances.

For example, JP-T-2020-514263 discloses a cosmetic composition comprising from 30% to 89% by weight of at least one polar oil relative to the total weight of the composition, from 10% to 45% by weight of at least one C₂-C₆ aliphatic monoalcohol relative to the total weight of the composition, and from 0.5% to 50% by weight of at least one polyol relative to the total weight of the composition, and at least one hydrophilic active agent, said composition comprising less than 7% by weight of water relative to the total weight of the composition.

However, there is a problem in that some cosmetic compositions comprising an oil and an alcohol are not stable. Therefore, there is still a demand for stable compositions comprising an oil and an alcohol for cosmetic use.

### DISCLOSURE OF INVENTION

An objective of the present invention is to provide to a stable composition comprising an oil and an alcohol.

The above objective of the present invention can be achieved by a composition, comprising:
(a) at least one fatty alcohol,
(b) at least one fatty acid ester oil,
(c) at least one C₂-C₄ diol, and
(d) water in an amount of 35% by weight or less relative to the total weight of the composition,
   wherein a total weight of oil(s) is less than 15% by weight relative to the total weight of the composition.

The (b) at least one fatty acid ester oil may be a monoester of the fatty acids and of a monoalcohol.

The (a) at least one fatty alcohol may be present in an amount ranging from 0.1% to 14% by weight, preferably from 0.3% to 10% by weight, more preferably from 0.5% to 7% by weight, and even more preferably from 0.7% to 5% by weight, relative to the total weight of the composition.

The (b) at least one fatty acid ester oil may be present in an amount of 0.5% to 14.5% by weight, preferably 1% to 12% by weight, more preferably from 2% to 10% by weight, and even more preferably 3% to 8% by weight, relative to the total weight of the composition.

The (c) at least one C₂-C₄ diol may be present in an amount of 1% to 20% by weight, preferably 2% to 15% by weight, more preferably 3% to 12% by weight, and even more preferably 5% to 10% by weight, relative to the total weight of the composition.

The amount of (d) water may be from 0.1% to 25% by weight, preferably from 1% to 20% by weight, more preferably from 3% to 15% by weight, and even more preferably from 5% to 10% by weight, relative to the total weight of the composition.

The total amount of the oil(s) is 1.5% to 14% by weight, preferably from 2.5% to 13% by weight, more preferably from 4% to 12% by weight, and even more preferably from 5% to 11% by weight, relative to the total weight of the composition.

The composition according to the present invention may further comprise at least one monoalcohol, preferably in an amount of 40% to 80% by weight, more preferably 45% to 70% by weight, and even more preferably 48% to 60% by weight, relative to the total weight of the composition.

The composition according to the present invention may further comprise at least one polyol distinct from the (c) at least one C₂-C₄ diol.

The at least one polyol may comprise a combination of at least one C₅-C₈ diol and at least one triol, preferably at least one C₅-C₈ diol in an amount ranging from 1% to 30% by weight and at least one triol in an amount ranging from 0.1% to 10% by weight, more preferably at least one C₅-C₈ diol in an amount ranging from 2% to 25% by weight and at least one triol in an amount ranging from 0.2% to 8% by weight, even more preferably at least one C₅-C₈ diol in an amount ranging from 4% to 20% by weight and at least one triol in an amount ranging from 0.4% to 5% by weight, and in particular at least one C₅-C₈ diol in an amount ranging from 8% to 15% by weight and at least one triol in an amount ranging from 0.8% to 3% by weight, relative to the total weight of the composition.

The composition according to the present invention may further comprise at least one hydrophilic active agent for the skin and/or scalp.

It is preferred that the composition according to the present invention comprise N-acylamino acid ester oil(s) in an amount of less than 1% by weight, preferably less than 0.5% by weight, more preferably less than 0.2% by weight, and even more preferably less than 0.1% by weight, relative to the total weight of the composition.

It is preferred that the composition according to present invention comprises surfactants, such as cationic, anionic, nonionic or amphoteric surfactants, in an amount of 5% by weight or less, preferably 3% by weight or less, and more preferably 2% by weight or less, relative to the total weight of the composition.

It one embodiment, the composition according to present invention is in the form of a homogeneous solution.

The present invention also relates to a cosmetic process for caring for and/or conditioning a keratin material, such as the skin, scalp and/or hair, comprising applying to the keratin material the composition according to the present invention.

### BEST MODE FOR CARRYING OUT THE INVENTION

After diligent research, the inventors have surprisingly discovered that the specific formulation of the composition comprising components (a) to (d), wherein a total weight of oil(s) is less than 15% by weight relative to the total weight of the composition, has an improved stability and a penetration property for an active ingredient for keratinous materials, while avoiding greasy feeling during application, and thus completed the present invention.

Thus, the present invention relates to a composition, comprising:
(a) at least one fatty alcohol,
(b) at least one fatty acid ester oil,
(c) at least one C₂-C₄ diol, and
(d) water in an amount of 35% by weight or less relative to the total weight of the composition,
   wherein a total weight of oil(s) is less than 15% by weight relative to the total weight of the composition.

Hereinafter, the cosmetic composition and cosmetic process according to the present invention will be explained in a more detailed manner.

### [Composition]

The composition according to the present invention includes (a) at least one fatty alcohol, (b) at least one fatty acid ester oil, (c) at least one C₂-C₄ diol, and (d) water. The ingredients in the composition will be described in a detailed manner below.

### (Fatty Alcohol)

The composition according to the present invention comprises (a) at least one fatty alcohol. Two or more types of fatty alcohols may be used in combination. Thus, a single type of fatty alcohol or a combination of different types of fatty alcohols may be used.

The term “fatty” in the fatty alcohol means the inclusion of a relatively large number of carbon atoms. Thus, alcohols which have 6 or more, preferably 8 or more, more preferably 10 or more, and even more preferably 12 or more carbon atoms are encompassed within the scope of fatty alcohols. The fatty alcohol may be saturated or unsaturated.

The fatty alcohol may have the structure R—OH wherein R is chosen from a saturated or unsaturated, linear or branched radical containing from 8 to 40 carbon atoms, preferably from 10 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

The fatty alcohol may have the structure R—OH wherein R is a saturated or unsaturated linear radical containing from 8 to 40 carbon atoms, preferably from 10 to 30 carbon atoms, and more preferably from 12 to 20 carbon atoms.

The fatty alcohol may have the structure R—OH wherein R is a saturated or unsaturated branched radical containing from 8 to 40 carbon atoms, preferably from 10 to 30 carbon atoms, and more preferably from 12 to 20 carbon atoms.

As examples of the fatty alcohol, mention may be made of lauryl alcohol, cetyl alcohol, stearyl alcohol, behenyl alcohol, isostearyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

In one embodiment of the present invention, the fatty alcohol is a saturated fatty alcohol. As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof.

In one embodiment of the present invention, the fatty alcohol is an unsaturated fatty alcohol. Thus, the fatty alcohol may be selected from straight or branched, unsaturated C₈-C₃₀ alcohols, preferably straight or branched, unsaturated C₁₀-C₂₆ alcohols, and more preferably straight or branched, unsaturated C₁₂-C₂₀ alcohols.

As examples of unsaturated fatty alcohols, mention may be made of oleyl alcohol, linoleyl alcohol, palmitoleyl alcohol, arachidonyl alcohol, erucyl alcohol, and mixtures thereof.

The fatty alcohol(s) may be present in the composition according to the present invention in an amount of 0.1% by weight or more, preferably 0.3% by weight or more, more preferably 0.5% by weight or more, and even more preferably 0.7% by weight or more, relative to the total weight of the composition.

The fatty alcohol(s) may be present in the composition according to the present invention in an amount of 14% by weight or less, preferably 10% by weight or less, more preferably 7% by weight or less, and even more preferably 5% by weight or more, relative to the total weight of the composition.

The fatty alcohol(s) may be present in the composition according to the present invention in an amount ranging from 0.1% to 14% by weight, preferably from 0.3% to 10% by weight, more preferably from 0.5% to 7% by weight, and even more preferably from 0.7% to 5% by weight, relative to the total weight of the composition.

### (Fatty Acid Ester Oil)

The composition according to the present invention comprises (b) at least one fatty acid ester oil. Two or more types of fatty acid ester oils may be used in combination. Thus, a single type of fatty acid ester oil or a combination of different types of fatty acid ester oils may be used.

The term “fatty acid ester” here is intended to mean an ester formed from at least one fatty acid and at least one alcohol.

The fatty acid ester oils may be liquid fatty acid esters of saturated or unsaturated, linear or branched C₄-C₃₀ fatty acids and of saturated or unsaturated, linear or branched C₁-C₂₆ aliphatic monoalcohols or polyalcohols, the total number of carbon atoms of the esters being greater than or equal to 10.

The fatty acid ester oils may be liquid esters of saturated linear C₄-C₂₆ fatty acid, preferably C₆-C₂₀ aliphatic monoacids, and more preferably C₁₀-C₂₀ aliphatic monoacids and of saturated branched C₁-C₂₀ aliphatic monoalcohols, preferably C₁-C₁₆ aliphatic monoalcohols, and even more preferably C₁-C₈ aliphatic monoalcohols the total number of carbon atoms of the esters being greater than or equal to 10.

The fatty acid ester oils of the monoesters of fatty acids and of monoalcohols may be represented by formula R₁COOR₂ in which R₁ represents the residue of a linear or branched, preferably a linear fatty acid comprising from 4 to 30 carbon atoms, preferably 8 to 24 carbon atoms, and more preferably 10 to 20 carbon atoms, and R₂ represents a hydrocarbon-based chain, especially branched, containing from 1 to 40 carbon atoms, preferably 1 to 12 carbon atoms, and more preferably 2 to 8 carbon atoms, with the proviso that R₁+R₂ is ≥10.

It is preferred that the alcohol of the fatty acid ester oils be branched.

Among the monoesters of fatty acids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate, and isostearyl neopentanoate.

The fatty acid ester oil may be chosen from the di and tri esters of saturated or unsaturated acid, preferably saturated acid, including 2 to 30 carbon atoms, preferably 6 to 30 carbon atoms, and more preferably 8 to 30 carbon atoms, such as lauric acid, oleic acid, stearic acid, isostearic acid, capric acid, caprylic acid, and myristic acid.

As the fatty acid ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides, or polysaccharides.

Examples of suitable sugars that may be mentioned include sucrose (or saccharose), glucose, galactose, ribose, fucose, maltose, fructose, mannose, arabinose, xylose, and lactose, and derivatives thereof, especially alkyl derivatives, such as methyl derivatives, for instance, methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters, and polyesters, and mixtures thereof.

These esters may be, for example, oleates, laurates, palmitates, myristates, behenates, cocoates, stearates, linoleates, linolenates, caprates, and arachidonates, or mixtures thereof such as, especially, oleopalmitate, oleostearate, and palmitostearate mixed esters, as well as pentaerythrityl tetraethyl hexanoate.

More particularly, use is made of monoesters and diesters and especially sucrose, glucose, or methylglucose monooleates or dioleates, stearates, behenates, oleopalmitates, linoleates, linolenates, and oleostearates.

An example that may be mentioned is the product sold under the name Glucate® DO by the company Amerchol, which is a methylglucose dioleate.

As examples of preferable fatty acid ester oils, mention may be made of, for example, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

The fatty acid ester oil(s) may be present in the composition according to the present invention in an amount of 0.5% by weight or more, preferably 1% by weight or more, more preferably 2% by weight or more, and even more preferably 3% by weight or more, relative to the total weight of the composition.

The fatty acid ester oil(s) may be present in the composition according to the present invention in an amount of 14.5% by weight or less, preferably 12% by weight or less, more preferably 10% by weight or less, and even more preferably 8% by weight or more, relative to the total weight of the composition.

The fatty acid ester oil(s) may be present in the composition according to the present invention in an amount ranging from 0.5% to 14.5% by weight, preferably from 1% to 12% by weight, more preferably from 2% to 10% by weight, and even more preferably from 3% to 8% by weight, relative to the total weight of the composition.

(C₂-C₄ Diol)

The composition according to the present invention comprises (c) at least one C₂-C₄ diol. Two or more types of C₂-C₄ diols may be used in combination. Thus, a single type of C₂-C₄ diol or a combination of different types of C₂-C₄ diols may be used.

The C₂-C₄ diol here means a diol having two to four carbon atoms. The C₂-C₄ diol that is suitable for use in the present invention may be a compound of linear or branched, saturated or unsaturated alkyl type, bearing two —OH functions on the alkyl chain.

Preferably, the C₂-C₄ diol that may be used in the composition according to the present invention is a compound of linear or branched, preferably linear and saturated alkyl type bearing two —OH functions on the alkyl chain.

The C₂-C₄ diol may be preferably chosen from ethylene glycol, propylene glycol, 1,3-propane diol, butylene glycol, 1,2-butane diol, 1,3-butane diol, 1,4-buthane diol, and mixtures thereof.

In a further preferred embodiment, the C₂-C₄ diol is selected from C₂-C₃ diols, such as ethylene glycol, propylene glycol and 1,3-propane diol.

In a specific embodiment, the C₂-C₄ diol is selected from C₃ diols, such as propylene glycol and 1,3-propane diol.

The C₂-C₄ diol(s) may be present in the composition according to the present invention in an amount of 1% by weight or more, preferably 2% by weight or more, more preferably 3% by weight or more, and even more preferably 5% by weight or more, relative to the total weight of the composition.

The C₂-C₄ diol(s) may be present in the composition according to the present invention in an amount of 20% by weight or less, preferably 15% by weight or less, more preferably 12% by weight or less, and even more preferably 10% by weight or more, relative to the total weight of the composition.

The C₂-C₄ diol(s) may be present in the composition according to the present invention in an amount ranging from 1% to 20% by weight, preferably from 2% to 15% by weight, more preferably from 3% to 12% by weight, and even more preferably from 5% to 10% by weight, relative to the total weight of the composition.

### (Water)

The composition according to the present invention can comprise water.

The amount of water in the composition according to the present invention may be 0.1% by weight or more, preferably 1% by weight or more, more preferably 3% by weight or more, and even more preferably 5% by weight or more, relative to the total weight of the composition.

The amount of water in the composition according to the present invention may be 35% by weight or less, preferably 25% by weight or less, more preferably 15% by weight or less, and even more preferably 10% by weight or less, relative to the total weight of the composition.

The amount of the water in the composition according to the present invention may be from 0.1% to 35% by weight, preferably from 1% to 25% by weight, more preferably from 3% to 15% by weight, and even more preferably from 5% to 10% by weight, relative to the total weight of the composition.

### (Oil)

In the composition according to the present invention, the total amount of oil(s) is less than 15% by weight relative to the total weight of the composition.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The (a) fatty alcohol and the (b) fatty acid ester oil are categorized into these oils. Therefore, the composition according to the present invention comprises oil(s) including the (a) at least one fatty alcohol and (b) at least one fatty acid ester oil in an mount less than 15% by weight relative to the total weight of the composition.

The composition according to the present invention may or may not comprise at least one additional oil in addition to the (a) fatty alcohol and the (b) fatty acid ester oil, as long as the total amount of the oil(s) is less than 15% by weight relative to the total weight of the composition.

Among the additional oils which may be used in the present invention, mention may be made of: volatile or non-volatile oils; these oils may be hydrocarbon-based oils, especially of animal or plant origin, synthetic oils, silicone oils, fluoro oils, or mixtures thereof.

For the purposes of the present invention, “hydrocarbon-based oil” or “hydrocarbon oil” is intended to mean an oil mainly containing hydrogen and carbon atoms and optionally oxygen, nitrogen, sulfur and/or phosphorus atoms. The hydrocarbon-based oil does not comprise any silicon atoms.

For the purposes of the present invention, “silicone oil” is intended to mean an oil comprising at least one silicon atom, and especially at least one Si—O group.

For the purposes of the present invention, “polar oil” is intended to mean an oil of which the solubility parameter Sa at 25° C. is other than 0 (J/cm³)^{1/2}.

In particular, “polar oil” is intended to mean an oil of which the chemical structure is formed essentially from, or even constituted of, carbon and hydrogen atoms, and comprising at least one highly electronegative heteroatom such as an oxygen, nitrogen, silicon or phosphorus atom.

The definition and calculation of the solubility parameters in the Hansen three-dimensional solubility space are described in the article by C. M. Hansen: The three-dimensional solubility parameters, J. Paint Technol., 39, 105 (1967).

According to this Hansen space:
δ_{D} characterizes the London dispersion forces resulting from the formation of dipoles induced during molecular impacts;
δₚ characterizes the Debye interaction forces between permanent dipoles and also the Keesom interaction forces between induced dipoles and permanent dipoles;
δₕ characterizes the forces of specific interactions (such as hydrogen bonds, acid/base bonds, donor/acceptor bonds, and the like);
δₐ is determined by the equation:

$${δ}_{a} = {( {δ}_{p}^{2} + {δ}_{h}^{2} )}^{1 / 2} .$$

The parameters δₚ, δₕ, δ_{D} and δₐ are expressed as (J/cm³)^{1/2}.

Preferably, the polar oils used according to the present invention have a δₐ of between 4 and 9.1, preferably a δₐ of between 6 and 9.1, even better still between 7.3 and 9.1.

The oil may be a non-polar oil such as a hydrocarbon oil, a silicone oil, or the like; a polar oil such as a plant or animal oil and an ether oil; or a mixture thereof.

The additional oil may be selected from the group consisting of oils of plant or animal origin, synthetic oils, silicone oils, and hydrocarbon oils.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ether oils, and artificial triglycerides.

As the ether oil, dialkyl ethers such as those represented by the following formula:

R¹—O—R²

wherein
each of R¹ and R² independently denotes a linear, branched or cyclic C₄-C₂₄ alkyl group, preferably C₆-C₁₈ alkyl group, and more preferably C₈-C₁₂ alkyl group. It is preferable that R¹ and R² are the same.

As the linear alkyl group, mention may be made of a butyl group, a pentyl group, a hexyl group, a heptyl group, an octyl group, a nonyl group, a decyl group, an undecyl group, a dodecyl group, tridecyl group, a tetradecyl group, a pentadecyl group, a hexadecyl group, a heptadecyl group, an octadecyl group, a nonadecyl group, an eicosyl group, a behenyl group, a docosyl group, a tricosyl group, and a tetracosyl group.

As the branched alkyl group, mention may be made of a 1-methylpropyl group, 2-methylpropyl group, a t-butyl group, a 1,1-dimethylpropyl group, a 3-methylhexyl group, a 5-methylhexyl group, an 1-ethylhexyl group, an 2-ethylhexylgroup, a 1-butylpentyl group, a 5-methyloctyl group, an 1-ethylhexyl group, an 2-ethylhexyl group, a 1-butylpentyl group, a 5-methyloctyl group, a 2-butyloctyl group, an isotridecyl group, a 2-pentylnonyl group, a 2-hexyldecyl group, an isostearyl group, a 2-heptylundecyl group, an 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyyl group, and a 2-(1,3,3-trimethylbutyl)-5,7,7-trimethyloctyl group.

As the cyclic alkyl group, mention may be made of a cyclohexyl group, a 3-methylcyclohexyl group, and a 3,3,5-trimethylcyclohexyl group.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate), and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of the formula:

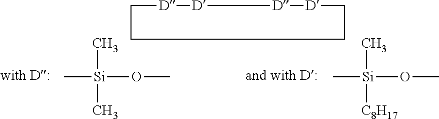

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluidsfor Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

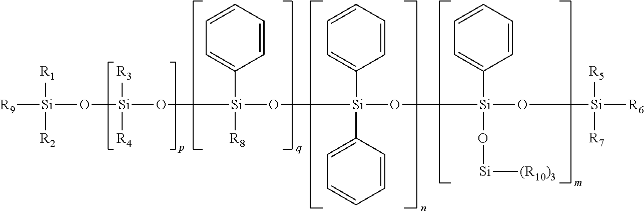

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl, or butyl radicals, and
m, n, p, and q are, independently of each other, integers of 0 to 900 inclusive, preferably 0 to 500 inclusive, and more preferably 0 to 100 inclusive,
with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250, and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

The hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane, and isodecane;
linear or branched hydrocarbons containing more than 16 carbon atoms, such as liquid paraffins, liquid petroleum jelly, polydecenes and hydrogenated polyisobutenes such as Parleam®, and squalane; and
mixtures of alkanes, for example, C9-12 Alkane, C10-13 Alkane, C13-14 Alkane, C13-15 Alkane, C14-17 Alkane, C14-19 Alkane, C15-19 Alkane, C15-23 Alkane, C18-21 Alkane, C8-9 Alkane/Cycloalkane, C9-10 Alkane/Cycloalkane, C9-11 Alkane/Cycloalkane, C9-16 Alkane/Cycloalkane, C10-12 Alkane/Cycloalkane, C11-14 Alkane/Cycloalkane, C11-15 Alkane/Cycloalkane, C12-13 Alkane/Cycloalkane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof. It is also preferable that the additional oil be chosen from oils with a molecular weight below 600 g/mol.

The total amount of the oil(s) including the (a) fatty alcohol and (b) fatty acid ester oil may be 14% by weight or less, preferably 13% by weight or less, more preferably 12% by weight or less, and even more preferably 11% by weight or less, relative to the total weight of the composition.

The total amount of the oil(s) including the (a) fatty alcohol and (b) fatty acid ester oil may be 1.5% by weight or more, preferably 2.5% by weight or more, more preferably 4% by weight or more, and even more preferably 5% by weight or more, relative to the total weight of the composition.

The total amount of the oil(s) including the (a) fatty alcohol and (b) fatty acid ester oil may be from 1.5% to 14% by weight, preferably from 2.5% to 13% by weight, more preferably from 4% to 12% by weight, and even more preferably from 5% to 11% by weight, relative to the total weight of the composition.

In one specific embodiment, the composition according to the present invention comprises N-acylamino acid ester oil(s) in an amount of less than 1% by weight, preferably less than 0.5% by weight, more preferably less than 0.2% by weight, and even more preferably less than 0.1% by weight, relative to the total weight of the composition. In a further specific embodiment according to the present invention, the composition is free from N-acylamino acid ester oil. As an example of N-acylamino acid ester oil, mention can be made of isopropyl lauroyl sarcosinate.

In another specific embodiment, the composition according to the present invention comprises ether oil(s) in an amount of less than 5% by weight, preferably less than 3% by weight, more preferably less than 2% by weight, and even more preferably less than 1% by weight, and in particular less than 0.5% by weight, relative to the total weight of the composition. In a further specific embodiment according to the present invention, the composition is free from ether oils.

### (Other Ingredients)

### Monoalcohol

The composition according to the present invention may comprise at least one monoalcohol. Two or more monoalcohols may be used in combination.

The monoalcohol may be water-soluble. The term “water-soluble alcohol” here means an alcohol which can dissolve in an amount of 0.1 g or more, 0.5 g or more, or 1 g or more in 100 mL of water at room temperature (25° C.) and atmospheric pressure (105 Pa).

The monoalcohol may be linear or branched, saturated or unsaturated mono-alcohols having from 1 to 8 carbon atoms, and preferably from 2 to 8 carbon atoms, bearing only one hydroxyl (OH) function.

In one embodiment, the monoalcohol may be aliphatic monoalcohol having from 1 to 8 carbon atoms, and preferably from 2 to 8 carbon atoms.

The term “aliphatic monoalcohol” here means any linear or branched, saturated alkane compound bearing only one hydroxyl (OH) function.

The aliphatic monoalcohol(s) present in the composition of the present invention may be chosen from ethanol, propanol, butanol, isopropanol, isobutanol and mixtures thereof.

In one preferred embodiment of the present invention, the monoalcohol can be selected from linear aliphatic monoalcohol having from 1 to 8 carbon atoms, and preferably from 2 to 8 carbon atoms, such as ethanol, propanol, butanol, and mixtures thereof.

The composition according to the present invention preferably comprises the monoalcohol(s), in particular ethanol, in an amount of 40% by weight or more, more preferably 45% by weight or more, and even more preferably 48% by weight or more; and preferably 80% by weight or less, more preferably 70% by weight or less, and even more preferably 60% by weight or less relative to the total weight of the composition.

The amount of monoalcohol may be from 40% to 80% by weight, preferably from 45% to 70% by weight, more preferably from 48% to 60% by weight, relative to the total weight of the composition.

Polyol Other than C₂-C₄ Diol

The composition according to the present invention may comprise at least one polyol. Two or more polyols may be used in combination.

For the purposes of the present invention, the term “polyol” should be understood as meaning any organic molecule comprising at least two free hydroxyl groups. The polyol here may be water-soluble.

The polyol that is suitable for use in the present invention may be a compound of linear, branched or cyclic, saturated or unsaturated alkyl type, bearing at least two —OH functions on the alkyl chain.

Preferably, a polyol that may be used in the composition according to the present invention is a compound of linear or branched, preferably linear alkyl type bearing at least two —OH functions, preferably 2 to 5 —OH functions, more preferably 2 to 4 —OH functions, and even more preferably 2 or 3 —OH functions on the alkyl chain.

The polyols that are advantageously suitable for formulating the cosmetic compositions according to the present invention are those especially having from 2 to 8 carbon atoms or for example 3 to 6 carbon atoms.

The polyols that may be used according to the present invention are chosen from linear or branched, preferably linear polyols having from 3 to 8 carbon atoms; mention may be made especially of:
C₅-C₈ diols such as hexylene glycol, dipropylene glycol, pentylene glycol, and isoprene glycol; and
triols, such as glycerol (glycerin), and mixtures thereof.

In one embodiment of the present invention, the composition comprises at least one C₅-C₈ diol in an amount of 1% by weight or more, preferably 2% by weight or more, more preferably 4% by weight or more, and even more preferably 8% by weight or more; and 30% by weight or less, preferably 25% by weight or less, more preferably 20% by weight or less; and even more preferably 15% by weight less, relative to the total weight of the composition.

In another embodiment of the present invention, the composition comprises at least one trio in an amount of 0.1% by weight or more, preferably 0.2% by weight or more, more preferably 0.4% by weight or more, and even more preferably 0.8% by weight or more; and 10% by weight or less, preferably 8% by weight or less, more preferably 5% by weight or less, and even more preferably 3% by weight less, relative to the total weight of the composition.

In one embodiment of the present invention, the polyol comprises a combination of at least one C₅-C₈ diol and at least one triol, preferably a C₅-C₈ diol in an amount ranging from 1% to 30% by weight and a triol in an amount ranging from 0.1% to 10% by weight, more preferably a C₅-C₈ diol in an amount ranging from 2% to 25% by weight and a triol in an amount ranging from 0.2% to 8% by weight, even more preferably a C₅-C₈ diol in an amount ranging from 4% to 20% by weight and a triol in an amount ranging from 0.4% to 5% by weight, and in particular e C₅-C₈ diol in an amount ranging from 8% to 15% by weight and a triol in an amount ranging from 0.8% to 3% by weight, relative to the total weight of the composition.

Polyol(s) other than C₂-C₄ diol(s) may be present in the composition according to the present invention in an amount ranging from 0.5% to 30% by weight, preferably from 1% to 20% by weight, and more preferably from 1.5% to 15% by weight, relative to the total weight of the composition.

### Hydrophilic Active Agent

The composition according to the present invention comprises (a) at least one hydrophilic active agent. Two or more hydrophilic active agents may be used in combination. Thus, a single type of hydrophilic active agent or a combination of different types of hydrophilic active agents may be used.

“Hydrophilic active agent” here is intended to mean an active agent for the scalp and/or skin, which is soluble in water at a concentration of at least 1% by weight relative to the total weight of the water at room temperature (25° C.) and atmosphere pressure (105 Pa).

As hydrophilic active agents, mention may be made, for example, of moisturizers; depigmenting agents; desquamating agents; anti-ageing agents; mattifying agents; cicatrizing agents; blood-circulation promoting ingredient; antibacterial agents; and mixtures thereof.

Preferably, said hydrophilic active agent is chosen from C-glycosides, salicylic acid, salicylic acid derivatives and mixtures thereof.

In a first embodiment, the composition according to the present invention may comprise at least one hydrophilic active agent chosen from C-glycosides of the following general formula (I):

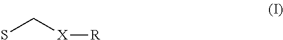

in which:
R denotes an unsubstituted linear C₁-C₄ and especially C₁-C₂ alkyl radical, in particular methyl;
S represents a monosaccharide chosen from D-glucose, D-xylose, N-acetyl-D-glucosamine and L-fucose, and in particular D-xylose;
X represents a group chosen from —CO—, —CH(OH)— and —CH(NH₂)— and preferentially a —CH(OH)— group;
and also the cosmetically acceptable salts thereof, solvates thereof such as hydrates, and optical isomers thereof.

As non-limiting illustrations of C-glycoside derivatives of formula (I) that are more particularly suitable for the present invention, mention may especially be made of the following compounds:
C-beta-D-xylopyranoside-n-propan-2-one;
C-alpha-D-xylopyranoside-n-propan-2-one;
C-beta-D-xylopyranoside-2-hydroxypropane;
C-alpha-D-xylopyranoside-2-hydroxypropane;
1-(C-beta-D-glucopyranosyl)-2-hydroxypropane;
1-(C-alpha-D-glucopyranosyl)-2-hydroxypropane;
1-(C-beta-D-glucopyranosyl)-2-aminopropane;
1-(C-alpha-D-glucopyranosyl)-2-aminopropane;
3′-(acetamido-C-beta-D-glucopyranosyl)propan-2′-one;
3′-(acetamido-C-alpha-D-glucopyranosyl)propan-2′-one;
1-(acetamido-C-beta-D-glucopyranosyl)-2-hydroxypropane;
1-(acetamido-C-beta-D-glucopyranosyl)-2-aminopropane;
and also the cosmetically acceptable salts thereof, solvates thereof such as hydrates, and optical isomers thereof.

According to a particular embodiment, use is made of C-beta-D-xylopyranoside-2-hydroxypropane or C-alpha-D-xylopyranoside-2-hydroxypropane, and better still C-beta-D-xylopyranoside-2-hydroxypropane.

According to a particular embodiment, a C-glycoside of formula (I) that is suitable for the invention may advantageously be C-beta-D-xylopyranoside-2-hydroxypropane, the INCI name of which is HYDROXYPROPYL TETRAHYDROPYR ANTRIOL, sold especially under the name MEXORYL SBB® or MEXORYL SCN® by NOVEAL. The salts of the C-glycosides of formula (I) that are suitable for the invention may comprise conventional physiologically acceptable salts of these compounds, such as those formed from organic or inorganic acids. Examples that may be mentioned include the salts of mineral acids, such as sulfuric acid, hydrochloric acid, hydrobromic acid, hydriodic acid, phosphoric acid and boric acid. Mention may also be made of the salts of organic acids, which may comprise one or more carboxylic, sulfonic or phosphonic acid groups. They may be linear, branched or cyclic aliphatic acids, or alternatively aromatic acids. These acids may also comprise one or more heteroatoms chosen from O and N, for example in the form of hydroxyl groups. Mention may especially be made of propionic acid, acetic acid, terephthalic acid, citric acid and tartaric acid.

The solvates that are acceptable for the compounds described above comprise conventional solvates such as those formed during the final step of preparation of said compounds due to the presence of solvents. Examples that may be mentioned include solvates due to the presence of water or of linear or branched alcohols, such as ethanol or isopropanol.

The C-glycosides (I) are also known from the document WO 02/051828.

In another particular embodiment, the composition according to the present invention may comprise at least one hydrophilic active agent chosen from salicylic acid and salicylic acid derivatives, in particular of formula (II below:

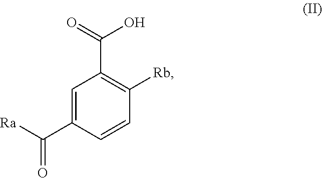

in which:
the radical Ra denotes:
a linear, branched or cyclic, saturated aliphatic chain containing from 2 to 22 carbon atoms;
an unsaturated chain containing from 2 to 22 carbon atoms, containing one or more double bonds which may be conjugated;
an aromatic nucleus bonded to the carbonyl radical directly or by means of saturated or unsaturated aliphatic chains containing from 2 to 7 carbon atoms;
it being possible for said groups to be substituted with one or more identical or different substituents chosen from:
(a) halogen atoms,
(b) the trifluoromethyl group,
(c) hydroxyl groups in free form or in a form esterified with an acid containing from 1 to 6 carbon atoms, or
(d) a carboxyl function in free form or in a form esterified with a lower alcohol containing from 1 to 6 carbon atoms;
Rb is a hydroxyl group;
   and also salts thereof derived from an inorganic or organic base.

According to one embodiment, the radical Ra denotes:
a linear, branched or cyclic, saturated aliphatic chain containing from 3 to 11 carbon atoms; or
an unsaturated chain containing from 3 to 17 carbon atoms and comprising one or more conjugated or non-conjugated double bonds;
it being possible for said hydrocarbon-based chains to be substituted with one or more identical or different substituents chosen from:
(a) halogen atoms;
(b) the trifluoromethyl group;
(c) hydroxyl groups in free form or in a form esterified with an acid containing from 1 to 6 carbon atoms; or
(d) a carboxyl function in free form or in a form esterified with a lower alcohol containing from 1 to 6 carbon atoms;
   and also salts thereof obtained by salification with an inorganic or organic base. The compounds that are more particularly preferred are those in which the radical Ra is a C₃-C₁₁ alkyl group.

Among the compounds of formula (II) that are particularly preferred, mention may be made of: 5-n-octanoylsalicylic acid (or capryloylsalicylic acid); 5-n-decanoylsalicylic acid; 5-n-dodecanoylsalicylic acid; 5-n-heptyloxysalicylic acid, and the corresponding salts thereof.

The salicylic acid compound is advantageously chosen from salicylic acid and 5-n-octanoylsalicylic acid. 5-n-Octanoylsalicylic acid will more particularly be used. 5-n-octanoylsalicyliic acid (or capryloylsalicylic acid) is provided under the name Mexoryl SAB® by the company Chimex.

Examples of inorganic bases that may be mentioned include alkali metal or alkaline-earth metal hydroxides, for instance sodium hydroxide, potassium hydroxide or aqueous ammonia. Among the organic bases that may be mentioned are amines and alkanolamines. Quaternary salts, for instance those described in patent FR 2 607 498, are particularly advantageous. The compounds of formula (II) that may be used according to the invention are described in patents U.S. Pat. Nos. 6,159,479, 5,558,871, FR 2 581 542, FR 2 607 498, U.S. Pat. No. 4,767,750, EP 378 936, U.S. Pat. Nos. 5,267,407, 5,667,789, 5,580,549 and EPA-570 230.

As another hydrophilic active agent, mention can be made of blood-circulation promoting ingredient, such as Dl-α-tocopherol acetate, tocopherol nicotinate, glucosyl hesperidin, hesperidin, caffeine, γ-oryzanol, capsaicin, nicotinic acid benzyl ester, and the like.

As another hydrophilic active agent, mention can be made of amino acid or its derivative, such as betain(trimethylglycine), proline, hydroxyproline, arginine, lysine, serine, glycine, alanine, phenylalanine, β-alanine, threonine, glutamic acid, glutamine, aspartic acid, cysteine, cystine, methionine, leucine, isoleucine, valine, histidine, threonine, tyrosine, taurine, γ-aminobutyric acid, γ-amino-β-hydroxybutyric acid, carnitine, camosine, creatine, epsilon aminocaproic acid, tryptophan, and the like.

As another hydrophilic active agent, mention can be made of water-soluble polysaccharides, such as gums, for example, gellan gum, xanthan gum, sclerothium gum, locust bean gum, biosaccharide gum, tamarind gum, quince seed, arabic gum, carrageenan, tara gum, guar gum, galactan, arabic gum, acacia gum, tragacanth gum, curdran, succinoglucan, etc.; carrageenan; heparin-like substances; alginic acids, and the like.

The hydrophilic active agent(s) may be present in the composition according to the present invention in an amount ranging from 0.1% to 20% by weight or more, from 0.5% to 15% by weight more preferably from 1% to 12% by weight, even more preferably from 2% to 10% by weight or more, and in particular from 3% to 10% by weight, relative to the total weight of the composition.

### Nonionic Surfactant

The composition according to the present invention may comprise at least one nonionic surfactant. Two or more nonionic surfactants may be used in combination.

Examples of nonionic surfactants usable in the compositions of the present invention include polyethoxylated fatty alcohols or polyglycerolated fatty alcohols, such as the adducts of ethylene oxide with lauryl alcohol, especially those containing from 9 to 50 oxyethylene units (Laureth-9 to Laureth-50 as the INCI names), in particular Laureth-9; esters of polyols and of a fatty acid possessing a saturated or unsaturated chain comprising, for example, from 8 to 24 carbon atoms, and their oxyalkylenated derivatives, that is to say comprising oxyethylene and/or oxypropylene units, such as esters of glycerol and of a C₈-C₂₄ fatty acid, and their oxyalkylenated derivatives, in particular polyoxyethylenated glyceryl stearate (mono-, di- and/or tristearate), for example PEG-20 glyceryl triisostearate; esters of sugar and of a C₈-C₂₄ fatty acid and their oxyalkylenated derivatives, such as polyethoxylated sorbitol esters of C₈-C₂₄ fatty acids, in particular Polysorbate 21, such as the product marketed under the name “TWEEN 21” by Croda; ethers of a sugar and of C₈-C₂₄ fatty alcohols, such as caprylyl/capryl glucoside; hydrophobized polysaccharides; polyoxyethylene alkyl ethers; polyoxyethylene oxypropylene alkyl ethers; fatty acid alkanol amides; alkyl amine oxides; alkyl polyglycosides and silicone surfactants, such as polydimethylsiloxane containing oxyethylene groups and/or oxypropylene groups, for example, PEG-10 dimethicone, bis-PEG/PPG-14/14 dimethicone, bis-PEG/PPG-20/20 dimethicone, and PEG/PPG-20/6 dimethicone; and polyglyceryl fatty acid ester such as polyglyceryl-4 caprate, polyglyceryl-10 laurate, polyglyceryl-6 dicaprate, polyglyceryl-6 dicaprate, polyglyceryl-6 dioleate, polyglyceryl-6 caprylate, polyglyceryl-2 oleate, and polyglyceryl-6 polyricinoleate; and mixtures thereof.

In addition, mention can be made of alkylpolyglycosides as nonionic surfactants, represented by the following general formula (1):

R—O-(G)ₓ (1)

in which R represents a branched and/or unsaturated alkyl radical comprising from 14 to 24 carbon atoms, G represents a reduced sugar comprising 5 or 6 carbon atoms and x denotes a value ranging from 1 to 10 and preferably from 1 to 4, and G in particular denotes glucose, fructose or galactose. Mention may be made, as alkyl polyglycosides of this type, of alkyl polyglucosides (G=glucose in the formula (I)) and in particular the compounds of formula (I) in which R more particularly represents an oleyl radical (unsaturated C₁₈ radical) or isostearyl (saturated C₁₈ radical), G denotes glucose and x is a value ranging from 1 to 2, in particular isostearyl glucoside, oleyl glucoside and their mixtures. This alkyl polyglucoside can be used as a mixture with a coemulsifier, more especially with a fatty alcohol and in particular a fatty alcohol having the same fatty chain as that of the alkyl polyglucoside, that is to say comprising from 14 to 24 carbon atoms and having a branched and/or unsaturated chain, for example isostearyl alcohol when the alkyl polyglucoside is isostearyl glucoside and oleyl alcohol when the alkyl polyglucoside is oleyl glucoside.

The nonionic surfactant(s) may be present in an amount of 0.1% by weight or more, preferably 0.5% by weight or more, more preferably 1% by weight or more, even more preferably 1.3% by weight or more, and in particular 1.5% by weight or more, and/or may be present in an amount of 10% by weight or less, preferably 7% by weight or less, more preferably 5% by weight or less, and in particular 3% by weight or less, relative to the total weight of the composition.

### Adjuvants

The composition according to the present invention may further comprise one or more of the adjuvants that are common in the fields of cosmetics and dermatology, selected from a physiologically acceptable medium, in particular water-soluble organic solvents; cationic, anionic, nonionic or amphoteric surfactants; cationic, anionic, non-ionic, amphoteric or zwitterionic polymers or mixtures thereof; gelling agents; thickeners; penetrating agents; pH adjuster; anti-dandruff agents; antioxidants; moisturizers; emollients; lipophilic active agents; free-radical scavengers; suspending agents; sequestering agents; buffer; fragrances; emollients; dispersing agents; dyes and/or pigments; film-forming agents; stabilizers; preservatives; co-preservatives; opacifying agents; and mixtures thereof.

The composition according to the present invention may further comprise plant extracts, such as extracts from Nymphaeaceous plants, for example, Nymphaea alba root extract.

The composition according to the present invention may further comprise nonionic guar gum, such as hydroxypropyl guar gum, in particular hydroxypropyl guar gum having more than 0.6 of degree of substitution. The term “degree of substitution” here is meant the average substitution of nonionic groups per anhydro sugar unit in the polygalactomannan gums. As commercially available products of the nonionic guar gum, mention can be made of hydroxypropyl guar gum sold under the name of Jaguar @ HSG by SOLVAY.

Of course, those skilled in the art will take care to select the optional adjuvant(s) added to the composition according to the invention such that the advantageous properties intrinsically associated with the composition in accordance with the invention are not, or are not substantially, adversely affected by the envisioned addition.

The adjuvants may be present in the composition of the present invention in an amount preferably ranging from 0.01% to 20% by weight, preferably from 0.1% to 10% by weight, and more preferably from 0.5% to 5% by weight, relative to the total weight of the composition.

The composition according to the present invention may take various forms, such as, a solution, a gel, a lotion, a serum, a suspension, a dispersion, a fluid, a milk, a paste, a cream, a foam, an emulsion (O/W or W/O form), multiple (e.g., W/0/W, polyol/O/W, and O/W/O) emulsions, and the like. In one specific embodiment, the composition according to the present invention is in the form of a solution, in particular a homogeneous solution. For the purposes of the present invention, the term “homogeneous” is intended to mean a composition consisting of a single phase.

In one specific embodiment of the present invention, the composition comprises surfactants, such as cationic, anionic, nonionic or amphoteric surfactants, which are different from components (a) to (c) of the present invention, in an amount of 5% by weight or less, preferably 3% by weight or less, and more preferably 2% by weight or less, relative to the total weight of the composition.

The composition according to the present invention is a cosmetic composition. In a particular embodiment, the composition according to the invention is intended for topical application, especially to keratin materials.

For the purposes of the present invention, “keratin materials” is intended to mean the skin and keratinous fibers. The term “skin” used herein includes facial and/or bodily skin and the scalp. The term “keratinous fiber” used herein include eyelashes, eyebrows, and hair.

According to a particular embodiment of the invention, the composition is intended for topical application to keratin materials such as the scalp and hair. In some embodiment, the composition may be a hair care and/or a scalp care and/or skin care composition.

The composition used according to the present invention is preferably intended to be used as a leave-on (or leave-in) type cosmetic composition. The term “leave-on” means a composition that is not intended to be washed/rinsed out or removed immediately after application. A leave-on composition is different from a rinse-off type composition, which is intended to be rinsed off after being used on keratin materials.

The composition according to the present invention can be prepared by mixing the above essential and optional ingredients in accordance with any of the processes which are well known to those skilled in the art.

### [Cosmetic Process and Use]

The present invention relates to a cosmetic process for caring for or conditioning a keratin material, such as the hair, scalp and/or skin, comprising applying to the keratin material the composition according to the present invention.

Cosmetic process here means a non-therapeutic cosmetic method for caring for and/or conditioning a keratin material.

The application step can be performed by any conventional means such as an applicator, e.g., hands, a spray, and a brush. The application step can be a topical application step.

The composition according to the present invention is intended to be used as a leave-on type cosmetic composition. Therefore, the cosmetic process according to the present invention do not include a rinse out or wash out step of the applied composition from the keratin material after the application step. In one embodiment of the present invention, the cosmetic process of the present invention does not include a rinse out or wash out step of the applied composition within one hour, preferably within 2 hours, more preferably within 4 hours, and even more preferably within 8 hours after the application step.

Moreover, the present invention also relates to a use of the composition according to the present invention in the field of cosmetics, in particular caring for and/or conditioning a keratin material.

### Examples

The present invention will be described in more detail by way of examples which however should not be construed as limiting the scope of the present invention.

### (Stability)

The compositions according to Example 1 and Comparative Examples 1 to 7 were prepared by mixing the ingredients listed in Table 1. Hydroxypropyl tetrahydropyrantriol product of MEXORYL SCN® was obtained from NOVEAL. The numerical values for the amounts of the ingredients shown in Table 1 are all based on “% by weight” as active raw materials.

10 g of each of obtained compositions was kept at 4° C., 25° C., and 45° C. for two months after they were manufactured, respectively. The stability property was evaluated for each composition under the following criteria.

OK: no phase separation was confirmed at any of 4° C., 25° C., and 45° C.

NG: the ingredient was not soluble or a phase separation occurred at 4° C., 25° C., and/or 45° C.

TABLE 1

| | | Comp. | Comp. | Comp. | Comp. | Comp. | Comp. |
|---|---|---|---|---|---|---|---|
| | Ex. 1 | Ex. 1 | Ex. 2 | Ex. 3 | Ex. 4 | Ex. 5 | Ex. 6 |
| Ingredient | | | | | | | |
| Ethanol | qs 100 | qs 100 | qs 100 | qs 100 | qs 100 | qs 100 | qs 100 |
| DI Water | 6.35 | 14.35 | 13.35 | 6.35 | 14.35 | 14.35 | 6.35 |
| Glycerin | 2 | 2 | 2 | 2 | 2 | 2 | 2 |
| Propylene Glycol | 8 | — | 8 | 8 | — | — | 8 |
| Pentylene Glycol | 12 | 12 | 12 | 12 | 12 | 12 | 12 |
| Hydroxypropyl | 8.7 | 8.7 | 8.7 | 8.7 | 8.7 | 8.7 | 8.7 |
| Tetrahydropyrantriol | | | | | | | |
| Polysorbate 21 | 1 | 1 | 1 | 1 | 1 | 1 | 1 |
| Isopropyl Lauroyl | — | — | 1 | 1 | 1 | 1 | 1 |
| Sarcosinate | | | | | | | |
| Oleyl Alcohol | 3.75 | 3.75 | 3.75 | 3.75 | 3.75 | 3.75 | 3.75 |
| Dicaprylyl Ether | — | — | — | 7 | 7 | — | 3.75 |
| Isopropyl Myristate | 7 | 7 | — | — | — | 7 | 7 |
| Menthol | 0.3 | 0.3 | 0.3 | 0.2 | 0.2 | 0.2 | 0.3 |
| Evaluation | | | | | | | |
| Stability | OK | NG | NG | NG | NG | NG | NG |

As can be seen from the results in Table 1, the compositions according to Examples 1 comprising ingredients (a) to (d) of the present invention, was stable. On the other hand, the compositions according to Comparative Examples 1 to 6 did not show good stability.

### (Penetration Analysis)

The compositions according to Example 1 and Comparative Examples 7 and 8 were evaluated with regard to the property of transdermal penetration of active ingredients in accordance with a Franz-cell (static diffusion cell) test as below. The formulation of compositions according to Comparative Examples 7 and 8 are shown in Tables 2 and 3 below, respectively. The numerical values for the amounts of the ingredients in Tables 2 and 3 are all based on “% by weight” as active raw materials. These compositions were prepared in the same manner as Example 1 above.

TABLE 2

| | Comp. Ex. 7 |
|---|---|
| Ingredients | (Prior Art) |
| Ethanol | qs 100 |
| Glycerin | 1.5 |
| Pentylene Glycol | 23.35 |
| Hydroxypropyl Tetrahydropyrantriol | 8.67 |
| Arginine | 0.1 |
| Oleyl Alcohol | 10 |
| Octyldodecanol | 29.88 |
| Ricinus Communis Seed Oil | 7.5 |
| Camelina Sativa Seed Oil | 4 |

TABLE 3

| Ingredients | Comp. Ex. 8 |
|---|---|
| Acacia Senegal Gum | 0.5 |
| Hydroxypropyl Tetrahydropyrantriol | 8.67 |
| Sodium Acetylated Hyaluronate | 0.1 |
| Pentylene Glycol | 12 |
| Propanediol | 2 |
| Propylene Glycol | 3.75 |
| Polysorbate 21 | 1 |
| Ethanol | 4.65 |
| Menthol | 0.3 |
| DI Water | qs 100 |

A porcine skin sample for the test was prepared by the following procedure. First, the skin of the outer side of porcine auricle was carefully separated from cartilage by a scalpel, and the porcine hair was carefully shaved to the same length by using a hair trimmer. After removal of the fat tissue under the skin, a dermatome (Zimmer®) was used to quantify the skin thickness to 750 μm and the experimental part without any damage was then carefully selected.

The composition according to each of Example 1 and Comparative Examples 7 and 8 was applied onto the skin sample with a spatula or a positive displacement pipette. The exact amount applied onto the skin was determined by weighing the spatula or positive displacement pipette containing the composition to be applied before and after the application so that the applied amount of the composition was about 5 mg/cm². The skin was dried in air for 2 minutes. The skin was then mounted onto a Franz cell. The Franz cell had an application area of 2 cm² and a cell receptor fluid was 3 mL of NaCl 0.9% (w/w) solution in water. The receptor fluid was stirred during the tests. The porcine skin is located between the donor compartment and the receptor compartment with the aforementioned application area of 2 cm². The temperature of the porcine skin was maintained to 32° C. After 16 hours, the skin surface was washed with an appropriate protocol to guarantee that all chemical remaining on the skin surface were removed, the porcine skin was removed, and then Stratum Corneum was removed by 10 consecutive tape strips (DSquam®).

Active ingredients (Hydroxypropyl Tetrahydropyrantriol) in the receptor fluid, epidermis, and dermis were analyzed with LC/MS. For each composition, three donors in triplicate (n=9) were used.

The mean value of the amount (g per cm² of the skin) of the active ingredient which passed through the Stratum Corneum of the porcine skin was calculated and is shown in Table 4.

TABLE 4

| | Ex. 1 | Com. Ex. 7 | Com. Ex. 8 |
|---|---|---|---|
| Penetration | 33.1 ± 20.2 | 38.2 ± 25.5 | 6.3 ± 2.1 |
| Amount (μg/cm²) | | | |

As can be seen from the results in Table 4, the use of the composition according to Example 1 showed a superior penetration property of the active ingredients.

### (Sensory Assessments)

The composition according to Example 2 was prepared by mixing the ingredients listed in Table 5. Hydroxypropyl tetrahydropyrantriol product of MEXORYL SCN® was obtained from NOVEAL. Hydroxypropyl guar gum products of Jaguar® HP 120 and Jaguar® HSG were obtained from Solvay. Nymphaea Alba Root Extract product of WATER LILY PRO® was obtained from LIPOID KOSMETIK. The numerical values for the amounts of the ingredients shown in Table 1 are all based on “% by weight” as active raw materials.

The composition according Example 2 was stable even after being kept at 25° C. for two months after it was manufactured, i.e. no phase separation was observed.

TABLE 5

| Ingredients | Ex. 2 |
|---|---|
| Ethanol | qs 100 |
| DI Water | 7.18 |
| Glycerin | 1.5 |
| Propylene Glycol | 7 |
| Nymphaea Alba Root Extract | 0.25 |
| Hydroxypropyl Tetrahydropyrantriol | 8.7 |
| Hydroxypropyl Guar Gum | 0.4 |
| Isostearyl Alcohol | 1 |
| Isopropyl Myristate | 5 |
| Menthol | 0.3 |
| Fragrance | 0.5 |

Three hair swatches (1 g, 27 cm) were prepared for each experiment. Each of the hair swatches was washed with a clarifying shampoo once. The formulation of the clarifying shampoo is shown in Table 6 below. After shampooing, the hair swatch was rinsed with water and then towel-dried. 0.2 g of each of the compositions according to Examples 1 and 2 and Comparative Example 7 was applied onto each hair swatch, and then the hair swatch was blow-dried to obtain a treated hair swatch.

TABLE 6

| Ingredients | Cleansing Shampoo |
|---|---|
| Hexylene Glycol | 1 |
| Sodium Chloride | 1 |
| Coco-Betaine | 9 |
| Sodium Laureth Sulfate | 15 |
| Polyquaternium-10 | 0.3 |
| Sodium Benzoate | 0.5 |
| Salicylic Acid | 0.2 |
| Citric Acid | qs pH 5.3 |
| Sodium Hydroxide | qs pH 5.3 |
| DI Water | qs 100 |

The greasiness sensation of the treated hair swatch were assessed by 6 panelists in accordance with the following criteria. The benchmark means the assessment result for the hair swatch itself, i.e., without the application of any composition.
5: Much higher than benchmark
4: Higher than benchmark
3: Parity to benchmark
2: Lower than benchmark
1: Much lower than benchmark

The scores thus obtained were averaged. The results are shown in Table 7. The composition according to Examples 1 and 2 were regarded as acceptable as topical cosmetic compositions in particular for the hair, skin, and scalp, by the panelists, while the compositions according to Comparative Example 7 was not regarded as acceptable.

TABLE 7

| | Ex. 1 | Ex. 2 | Com. Ex. 7 |
|---|---|---|---|
| Greasiness | 3.5 | 2.25 | 5.0 |

As can be seen from the results in Table 7, the compositions according to Examples 1 and 2 could impart a better texture, i.e. less grease texture, to the hair.

Therefore, it can be concluded that the composition according to the present invention is very suitable as a cosmetic composition, in particular a cosmetic topical composition for keratinous materials. In particular, the composition according to the present invention is very useful as a leave-on topical cosmetic composition since it exhibits a less greasy texture and better penetration property of the active ingredients. Accordingly, it can be said that the composition according to the present invention are very useful for topical delivery of active ingredients for keratinous substances, such as the skin and/or scalp.

1. A composition comprising:
(a) at least one fatty alcohol,
(b) at least one fatty acid ester oil,
(c) at least one C₂-C₄ diol,
(d) water in an amount of 35% by weight or less relative to the total weight of the composition,
wherein a total amount of oil(s) is less than 15% by weight relative to the total weight of the composition.

2. The composition according to claim 1, wherein the (b) at least one fatty acid ester oil is a monoester of fatty acids and of a monoalcohol.

3. The composition according to claim 1, wherein the (a) at least one fatty alcohol is present in an amount ranging from 0.1% to 14% by weighty relative to the total weight of the composition.

4. The composition according to claim 1, wherein the (b) at least one fatty acid ester oil is present in an amount of 0.5% to 14.5% by weight relative to the total weight of the composition.

5. The composition according to claim 1, wherein the (c) at least one C₂-C₄ diol is present in an amount of 1% to 20% by weight relative to the total weight of the composition.

6. The composition according to claim 1, wherein the amount of (d) water is from 0.1% to 25% by weight relative to the total weight of the composition.

7. The composition according to claim 1, wherein the total amount of the oil(s) is 1.5% to 14% by weight relative to the total weight of the composition.

8. The composition according to claim 1, further comprising at least one monoalcohol.

9. The composition according to claim 1, further comprising at least one polyol distinct from the (c) at least one C₂-C₄ diol.

10. The composition according to claim 9, wherein the at least one polyol comprises a combination of at least one C₅-C₈ diol and at least one triol.

11. The composition according to claim 1, further comprising at least one hydrophilic active agent for skin and/or scalp.

12. The composition according to claim 1, comprising N-acylamino acid ester oil(s) in an amount of less than 1% by weight relative to the total weight of the composition.

13. The composition according to claim 1, comprising surfactants in an amount of 5% by weight or less relative to the total weight of the composition.

14. The composition according to claim 1, which is in a form of a homogeneous solution.

15. A cosmetic process for caring for and/or conditioning a keratin material, comprising applying to the keratin material the composition according to claim 1.
